(19) United States
(12) Patent Application Publication
(10) Pub. No.: US 2024/0115503 A1
(43) Pub. Date: Apr. 11, 2024

(54) INTRAVESICAL DELIVERY OF HYDROPHILIC THERAPEUTIC AGENTS USING LIPOSOMES

(71) Applicant: Lipella Pharmaceuticals Inc., Pittsburgh, PA (US)

(72) Inventors: Jason Hafron, Pittsburgh, PA (US); Michael B. Chancellor, Pittsburgh, PA (US); Jonathan H. Kaufman, Pittsburgh, PA (US)

(21) Appl. No.: 18/011,635

(22) PCT Filed: Nov. 11, 2022
(86) PCT No.: PCT/US2022/079695

§ 371 Date: Dec. 20, 2022

Related U.S. Application Data
(60) Provisional application No. 63/280,426, filed on Nov. 17, 2021.

(51) Int. Cl. A61K 9/127; A61K 9/00; A61K 9/19

(52) U.S. Cl. CPC A61K 9/1277; A61K 9/0034; A61K 9/19

(57) ABSTRACT

Liposomal formulations for delivery of hydrophilic agents to a tissue or tissue lumen, such the bladder, are described herein. These liposomal formulations can be administered to a subject in need thereof by various means, such as by instillation, to treat a tissue or tissue lumen, such as of the bladder.

### CROSS-REFERENCE TO RELATED APPLICATION

This application claims the benefit of and priority to U.S. Provisional Application No. 63/280,426 filed Nov. 17, 2021, which is hereby incorporated by reference in its entirety.

### FIELD OF THE INVENTION

The invention is generally in the field of pharmaceutical formulations containing liposomes and therapeutic or prophylactic agents for the treatment or prevention of conditions, such as of the bladder, including hemorrhagic cystitis, cancer, and interstitial cystitis/painful bladder syndrome.

### BACKGROUND OF THE INVENTION

Pure sphingomyelin liposomes are known to be effective for the intravesical delivery of pharmaceutical formulations of hydrophobic agents. Delivery of hydrophilic agents via liposomes, however, remains a challenge.

There are issues and challenges in the treatment of bladder cancer. Approximately 70% of all newly diagnosed bladder tumors are non-muscle invasive bladder cancers (NMIBC), including stage Ta, stage T1 and carcinoma in situ (CIS). Non-muscle invasive bladder cancers exist on a continuum of risk in patients with T1 high-grade (T1Hg) bladder cancer at the aggressive end of the spectrum. Following transurethral resection alone, T1Hg bladder cancer has a 69% to 80% recurrence rate and a 33% to 48% chance of progression to muscle-invasive disease (Nepple et al, Can Urol Assoc J. 2009 December; 3(6 Suppl 4): S188-S192.)

Standard therapy for high-risk non-muscle invasive bladder cancer patients includes trans-urethral resection of the bladder tumor (TURBT) followed by Bacillus Calmette-Guerin (BCG) induction and maintenance. For patients with HG Ta, CIS, or recurrent T1 disease that have not responded to the first induction, a second induction may be given, while BCG therapy may be successful at preventing early tumor recurrences, most patients do not maintain sustained remissions. Despite TURBT and intravesical treatments, including BCG, approximately 40% of patients progress to muscle invasive disease. Progression to metastatic disease can occur in 20-30% of these individuals with death due to bladder cancer in nearly all of these patients (Cookson, M. S., et al., 1997 J Urol 158(1):62-7; and Millan-Rodriguez, F., et al., 2000 J Urol 64(3 Pt 1):680-4).

As an example, currently available intravesical approaches for the delivery of pharmaceutical immunoglobulin formulations consist solely of the “off-label” non-approved use of existing pure-water formulations manufactured for systemic intravenous injection. Currently, systemic PD-1/PD-L1 administration is being investigated in clinical trials in combination with intravesical BCG instillation at the same clinical session.

Accordingly, there is a need to provide means for delivery of hydrophilic agents via sphingomyelin liposomes which could have significant clinical utility.

It is another object of the invention to provide liposomes that provide improved delivery of hydrophilic therapeutic agents, and liposomal formulations thereof.

It is a further object of the invention to provide methods of treating patients in need thereof by, for example, direct application to a tissue or a tissue lumen, such as of the bladder, of a liposomal formulation delivering therapeutic or prophylactic agent.

### SUMMARY OF THE INVENTION

Liposomes having an outer phospholipid shell formed of, sphingophospholipids, such as sphingomyelin or a sphingomyelin metabolite, have been discovered to be particularly useful for delivery to the bladder of hydrophilic therapeutic agent(s), such as biologics like PD-1 inhibitors, such as PD-1 antibodies, for treatment of cancer. Sphingomyelin metabolites which can be used to form the liposomes can include ceramide, sphingosine, or sphingosine 1-phosphate. In preferred embodiments, the liposome is formed of sphingomyelin and/or a metabolite thereof, but may include one or more other lipids such as cholesterol. In these embodiments, the molar ratio of the sphingophospholipid, such as sphingomyelin, to a second lipid, such as cholesterol, present in the liposomes, is between about 5:1 to about 1:1 or 3:1 to about 1:1, more preferably from about 1.5:1 to about 1:1. In some instances, the molar ratio is about 1:1. The liposomes typically have an aqueous core.

### BRIEF DESCRIPTION OF THE DRAWINGS

FIG. 1 is a schematic of a solution 10 of sphingomyelin (SM) and cholesterol (CH) in a miscible mixture of tertiary butyl alcohol (TBA) and water (H₂O) on the left and on the right an aliquot 12 of solution 10 in a single-dose container 14.

FIG. 2 is a schematic of the transition of an aliquoted solution 12 in a single-dose container 14 via a lyophilization step to afford a pre-liposomal lyophilisate 16 in a vacuum-sealed container 14.

FIG. 3 is a schematic of the rehydration of a vacuum-sealed lyophilisate 16 in a vacuum-sealed container 18 by addition of an aqueously dissolved immunoglobulin 20, such as an anti-PD-1, resulting in formation of a liposomal immunoglobulin suspension 22 having an optimized immunoglobulin association with respect to the resulting therapeutic immunoglobulin potency.

### DETAILED DESCRIPTION OF THE INVENTION

### I. Definitions

“Agent” as used herein refers to a physiologically or pharmacologically active substance that acts locally and/or systemically in the body. An agent is a substance that is administered to a patient for the treatment (e.g., therapeutic agent), prevention (e.g., prophylactic agent), or diagnosis (e.g., diagnostic agent) of a disease or disorder.

“Hydrophobic” as used herein refers to a typically non-polar molecule or part of a molecule that cannot form energetically favorable interactions with water molecules and therefore does not dissolve in water, as compared to organic solvents.

“Hydrophilic” as used herein, refers to molecule(s) which have a greater affinity for, and thus solubility in, water as compared to organic solvents. For example, the hydrophilicity of a compound or molecule can be quantified by measuring its partition coefficient between water (or a buffered aqueous solution) and a water-immiscible organic solvent, such as octanol, ethyl acetate, methylene chloride, or methyl tert-butyl ether. If after equilibration a greater concentration of the compound or molecule is present in the water than in the organic solvent, then the compound or molecule is considered hydrophilic.

“Amphiphilic” as used herein describes a molecule having both hydrophobic and hydrophilic regions, as in a phospholipid or a detergent molecule.

“Effective amount” or “suitable amount” as used herein is at least the minimum concentration required to effect a measurable improvement or prevention of any symptom or a particular condition or disorder, to effect a measurable enhancement of life expectancy, or to generally improve patient quality of life. The effective amount is thus dependent upon the specific biologically active molecule and the specific condition or disorder to be treated. Effective amounts of many proteins, such as monoclonal antibodies (mAbs), are well known in the art. The effective amounts of proteins hereinafter discovered or for treating specific disorders with known proteins, such as mAbs, to be clinically applied to treat additional disorders may be determined by standard techniques which are well within the craft of a skilled artisan, such as a physician.

“Pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

“Solvent” as used herein refers to a liquid substance capable of dissolving other substances.

“Shell” as used herein refers to a deformable boundary of a three-dimensional object that maintains a constant surface area, but not necessarily a constant void volume, during deformation.

“Void” as used herein refers to the three-dimensional space within a shell.

“Volume” as used herein refers to the amount of three-dimensional space an object occupies.

“Void volume” as used herein refers to the volume of a void that is associated with a shell.

“Conformation of a shell” as used herein refers to the shape of a shell.

“Phospholipid shell” as used herein refers to a collection of phospholipids in the form of a shell that results from the interaction of phospholipids and an aqueous (or polar) solvent.

“Liposome” as used herein refers to a particle that is composed of one or more connected and/or concentric phospholipid shells.

“Planar projection of a liposome” as used herein refers to the linear mapping of all points of a liposome to corresponding points on a two-dimensional plane such that all lines connecting liposome points to their corresponding projection points are parallel to each other and perpendicular to the projection plane.

“Projection diameter of a liposome” as used herein refers to the diameter of a circle of a size such that it has an area equal to the mean of the areas of all of the liposome's possible planar projections.

“Membrane associated agent” as used herein refers to an agent, such as a drug, that preferentially partition within or adjacent to a biological membrane versus the membrane's surrounding aqueous solvent.

Numerical ranges disclosed herein include, but are not limited to, ranges of concentrations, ranges of integers, ranges of percentages, ranges of times, and ranges of size, etc. The disclosed ranges of any type, disclose individually each possible number that such a range could reasonably encompass, as well as any sub-ranges and combinations of sub-ranges encompassed therein. For example, disclosure of a time range is intended to disclose individually every possible time value that such a range could encompass, consistent with the disclosure herein.

Use of the term “about” is intended to describe values either above or below the stated value, which the term “about” modifies, in a range of approx. +/−10%; in other instances the values may range in value either above or below the stated value in a range of approx. +/−5%. When the term “about” is used before a range of numbers (i.e., about 1-5) or before a series of numbers (i.e., about 1, 2, 3, 4, etc.) it is intended to modify both ends of the range of numbers and/or each of the numbers recited in the entire series, unless specified otherwise.

### II. Liposome Containing Formulations

Liposomes in combination with pharmaceutical agents for treating patients in need thereof may be used to form formulations with one or more excipients. The formulations can be in the form of a liquid, powder, or gel.

A. Liposomes

A liposome is composed of one or more connected and/or concentric phospholipid shells (Torchilin and Weissig, Liposomes, Second Edition, Oxford University Press (2003)). Liposomes are spherical vesicles composed of concentric phospholipid bilayers separated by aqueous compartments. Liposomes can adhere to and form a molecular film on cellular surfaces. Structurally, liposomes are lipid vesicles composed of concentric phospholipid bilayers which enclose an aqueous interior (Gregoriadis, et al., Int. J. Pharm., 300, 125-30 2005; Gregoriadis and Ryman, Biochem. J., 124, 58P (1971)). Hydrophobic compounds associate with the lipid phase, while hydrophilic compounds associate with the aqueous phase.

In preferred embodiments, sphingophospholipids, such as sphingomyelin, are used to form the phospholipid shell of the liposomes. The liposomes may also include a sphingomyelin metabolite. Sphingomyelin metabolites which can be used to form the liposomes include ceramide, sphingosine, or sphingosine 1-phosphate.

In certain embodiments, the liposomes are generated from a single type of sphingophospholipid, such as sphingomyelin. In other embodiments, the lipids may be formed from a combination of more than one lipid. Such liposomes may include one or more additional lipids, which can be neutral, anionic, or cationic at physiologic pH. Suitable neutral and anionic lipids include sterols and lipids such as cholesterol, phospholipids, lysolipids, lysophospholipids, sphingolipids or pegylated lipids. Neutral and anionic lipids include phosphatidylcholine (PC) (such as egg PC, soy PC), including 1,2-diacyl-glycero-3-phosphocholines; phosphatidylserine (PS), phosphatidylglycerol, phosphatidylinositol (PI); glycolipids; sphingoglycolipids (also known as 1-ceramidyl glucosides) such as ceramide galactopyranoside, gangliosides and cerebrosides; fatty acids, sterols containing a carboxylic acid group, for example, cholesterol; 1,2-diacyl-sn-glycero-3-phosphoethanolamine, including, but not limited to, 1,2-dioleylphosphoethanolamine (DOPE), 1,2-dihexadecylphosphoethanol amine (DHPE), 1,2-distearoylphosphatidylcholine (DSPC), 1,2-dipalmitoyl phosphatidylcholine (DPPC), and 1,2-dimyristoylphosphatidylcholine (DMPC). The lipids can also include various natural (e.g., tissue derived L-α-phosphatidyl: egg yolk, heart, brain, liver, soybean) and/or synthetic (e.g., saturated and unsaturated 1,2-diacyl-sn-glycero-3-phosphocholines, 1-acyl-2-acyl-sn-glycero-3-phosphocholines, 1,2-diheptanoyl-SN-glycero-3-phosphocholine) derivatives of the lipids. In some instances, the liposomes including a second lipid, preferably a sterol such as cholesterol.

In some cases, the concentration of the sphingophospholipids, such as sphingomyelin and/or metabolites thereof, used to formulate the liposomes can be at any suitable concentration. In some instances, the concentration in a liposomal formulation is between about 0.1 mol % to about 100 mol %, 0.1 mol % to about 90 mol %, 0.1 mol % to about 80 mol %, 0.1 mol % to about 70 mol %, 0.1 mol % to about 60 mol %, 0.1 mol % to about 50 mol %, about 0.1 mol % to about 40 mol %, about 0.1 mol % to about 30 mol %, about 0.1 mol % to about 20 mol %, about 0.1 mol % to about 10 mol %, about 2.0 mol % to about 5.0 mol %, or from about 1.0 mol %; and sub-ranges therein. In some cases, such as when a dry powdered pre-liposomal lyophilisate is used to form the liposomes on re-hydration of the pre-liposomal lyophilisate powder, the concentration of the sphingophospholipids, such as sphingomyelin and/or metabolites thereof, within the pre-liposomal lyophilisate used to formulate the liposomes in the formulation is between about from 0.1 mol % to about 100 mol % or any concentration sub-range thereof.

In some instances, the molar ratio of the sphingophospholipid, such as sphingomyelin, to a second lipid, such as cholesterol, present in the liposomes, is between about 5:1 to about 1:1 or 3:1 to about 1:1, more preferably from about 1.5:1 to about 1:1. In some instances, the molar ratio is about 1:1.

The liposomes typically have an aqueous core. Preferably, in some instances, the aqueous core contains a mixture of water and an alcohol. Suitable alcohols include, but are not limited to, methanol, ethanol, propanol, (such as isopropanol), butanol (such as n-butanol, isobutene, sec-butanol, tert-butanol, pentanol (such as amyl alcohol, isobutyl carbinol), hexanol (such as 1-hexanol, 2-hexanol, 3-hexanol), heptanol (such as 1-heptanol, 2-heptanol, 3-heptanol and 4-heptanol) or octanol (such as 1-octanol) or a combination thereof.

The liposomes have either one or several aqueous compartments delineated by either one (unilamellar) or several (multilamellar) phospholipid bilayers (Sapra, et al., Curr. Drug Deliv., 2, 369-81 (2005)). Preferably, the liposomal formulations contain liposomes forming from 1 to 100% of the liposome population in the formulation. In some embodiments, large liposomes represent greater than approximately 50% of the liposome population in the formulation.

In some instances, the liposomes are formed by rehydrating and/or reconstituting a pre-liposomal lyophilisate made by lyophilizing a solution of a sphingophospholipid, such as sphingomyelin, and a surfactant, such as cholesterol, in a water/tert-butyl alcohol co-solvent system. When an aqueous solution containing agent(s) is combined with the pre-liposomal lyophilisate, a liposomal formulation containing liposomes having the agents associated therewith is formed. Details on methods of forming the pre-liposomal lyophilisate and forming the liposomal formulations is provided in the methods discussed below.

In some instances, the liposomes formed by rehydrating and/or reconstituting a pre-liposomal lyophilisate and found in the formulations described have an average diameter ranging from between about 0.1 to 50 microns, and any sub-ranges disclosed within. Preferably, the average diameter of the liposomes in the formulations will range from between about 10 to about 900 nanometers, about 10 to about 800 nanometers, about 10 to about 700 nanometers, about 10 to about 600 nanometers, about 10 to about 500 nanometers, about 10 to about 400 nanometers, about 10 to about 300 nanometers, about 10 to about 200 nanometers, or about 10 to about 100 nanometers, and any suitable sub-ranges within those mentioned, so that the small size of the liposomes reduces, prevents, or eliminates the likelihood of the liposomes in the formulation from settling, as determined by visual inspection, within a period of at least four hours at standard pressure and temperature.

B. Therapeutic, Prophylactic and Diagnostic Agents Agents that can be delivered via the liposomal formulations include one or more therapeutic, prophylactic, or diagnostic agents. These may be small molecules, sugars, polysaccharides, nucleotides, oligonucleotides, lipids, lipoproteins, proteins, and peptides.

The ratio of therapeutic, prophylactic, or diagnostic agent to lipid(s) (International units or weight, micrograms or milligrams, of active agent per mg of lipid(s)) can be varied to regulate the amount of agent that is released, and over what time period. Suitable agent to lipid(s) ratios include, but are not limited to, 1:1, 1:0.9, 1:0.8, 1:0.7, 1:0.6, 1:0.5, 1:0.4, 1:0.3, 1:0.2, or 1:0.1 (activity unit or weight of active agent per mg of lipid).

In a preferred embodiment, the liposomes include a hydrophilic agent, an immunoglobulin such as an anti-PD-1 (programmed cell death protein 1) immune checkpoint inhibitor, for treating bladder diseases, such as cancer. Programmed cell death-1 includes programmed cell death-1 (PD-1)/programmed death-ligand 1 (PD-L1) checkpoint inhibitors in PD-1/PD-L1 signaling pathways. Other hydrophilic therapeutic, prophylactic, or diagnostic agents known in the art may also be used and included in the liposomes described, as would be understood by the skilled person.

Other agents which may be included, such as inhibitory nucleic acids, include, but not limited to, ribozymes, triplex-forming oligonucleotides (TFOs), antisense DNA, non-enveloped (naked) RNA, siRNA, and microRNA specific for nucleic acids encoding the chemokines. The antisense DNA oligonucleotides typically include at least 18, 19, 20, 21, 22, 23, 24 or 25 nucleotides and are preferably at least 20 nucleotides in length. Inhibitory nucleic acids and methods of producing them are well known in the art. siRNA design software is available, for example, at http://i.cs.hku.hk/˜sirna/software/sirna.php. Synthesis of nucleic acids is well known, see, for example, Molecular Cloning: A Laboratory Manual (Sambrook and Russel eds. 3^{rd} ed.) Cold Spring Harbor, New York (2001).

The term “siRNA” means a small interfering RNA that is a short-length double-stranded RNA that is not toxic. Generally, there is no particular limitation of the length of siRNA as long as it does not show toxicity. “siRNAs” can be, for example, 15 to 49 bp, preferably 15 to 35 bp, and more preferably 21 to 30 bp long. Alternatively, the double-stranded RNA portion of a final transcription product of siRNA to be expressed can be, for example, 15 to 49 bp, preferably 15 to 35 bp, and more preferably 21 to 30 bp long. In a preferred embodiment, the siRNA is at least 19, 20, 21, 22, or 23 nucleotides long. The double-stranded RNA portions of siRNAs in which two RNA strands pair up are not limited to the completely paired ones, and may contain nonpairing portions due to mismatch (the corresponding nucleotides are not complementary), or bulge (lacking in the corresponding complementary nucleotide on one strand). Non-pairing portions can be contained to the extent that they do not interfere with siRNA formation. The “bulge” preferably comprise 1 to 2 nonpairing nucleotides, and the double-stranded RNA region of siRNAs in which two RNA strands pair up contains preferably 1 to 7, more preferably 1 to 5 bulges. In addition, the “mismatch” used herein is contained in the double-stranded RNA region of siRNAs in which two RNA strands pair up, preferably 1 to 7, more preferably 1 to 5, in number. In a preferable mismatch, one of the nucleotides is guanine, and the other is uracil. Such a mismatch is due to a mutation from C to T, G to A, or mixtures thereof in DNA coding for sense RNA, but not particularly limited to them. Furthermore, the double-stranded RNA region of siRNAs in which two RNA strands pair up may contain both bulge and mismatched, which sum up to, preferably 1 to 7, more preferably 1 to 5 in number.

The terminal structure of siRNA may be either blunt or cohesive (overhanging) as long as siRNA can silence, reduce, or inhibit the target gene expression due to its RNAi effect. The cohesive (overhanging) end structure is not limited only to the 3′ overhang, and the 5′ overhanging structure may be included as long as it is capable of inducing the RNAi effect. In addition, the number of overhanging nucleotide is not limited to the already reported 2 or 3, but can be any numbers as long as the overhang is capable of inducing the RNAi effect. For example, the overhang consists of 1 to 8, preferably 2 to 4 nucleotides. Herein, the total length of siRNA having cohesive end structure is expressed as the sum of the length of the paired double-stranded portion and that of a pair comprising overhanging single-strands at both ends. For example, in the case of 19 bp double-stranded RNA portion with 4 nucleotide overhangs at both ends, the total length is expressed as 23 bp. Furthermore, since this overhanging sequence has low specificity to a target gene, it is not necessarily complementary (antisense) or identical (sense) to the target gene sequence. Furthermore, as long as siRNA is able to maintain its gene silencing effect on the target gene, siRNA may contain a low molecular weight RNA (which may be a natural RNA molecule such as tRNA, rRNA or viral RNA, or an artificial RNA molecule), for example, in the overhanging portion at its one end. In addition, the terminal structure of the siRNA is not necessarily the cut off structure at both ends as described above, and may have a stem-loop structure in which ends of one side of double-stranded RNA are connected by a linker RNA. The length of the double-stranded RNA region (stem-loop portion) can be, for example, 15 to 49 bp, preferably 15 to 35 bp, and more preferably 21 to 30 bp long. Alternatively, the length of the double-stranded RNA region that is a final transcription product of siRNAs to be expressed is, for example, 15 to 49 bp, preferably 15 to 35 bp, and more preferably 21 to 30 bp long. Furthermore, there is no particular limitation in the length of the linker as long as it has a length so as not to hinder the pairing of the stem portion. miRNAs are produced by the cleavage of short stem-loop precursors by Dicer-like enzymes; whereas, siRNAs are produced by the cleavage of long double-stranded RNA molecules. miRNAs are single-stranded, whereas siRNAs are double-stranded. Methods for producing miRNA are known in the art. Because the sequences for CCL2 (MCP-1), CCL4 (MIP-1β), CCL11 (eotaxin), CXCL1 (GRO-α), sCD40L, IL-12p70/p40, IL-5, sIL-2Rα, IL-6, IL-10, IL-8, and EGF are known, one of skill in the art could readily produce miRNAs that downregulate expression of these chemokines using information that is publicly available.

Increasing the biological activity of growth factors relevant to urological disorders is effective to treat certain urological disorders, in particular interstitial cystitis/painful bladder syndrome and overactive bladder syndrome. The presence of elevated levels of EGF in urine of patients with overactive bladder syndrome is suggestive of tissue repair and fibrosis. An effective amount of one or more growth factors to diminish the severity or number of symptoms of a urological disorder is administered to a subject having one or more symptoms of a urological disorder. Preferred growth factors include, but are not limited to, vascular endothelial growth factor (VEGF), bone morphogenetic protein (BMP), a transforming growth factor (TGF) such as transforming growth factor, a platelet derived growth factor (PDGF), an epidermal growth factor (EGF), a nerve growth factor (NGF), an insulin-like growth factor (e.g., insulin-like growth factor I), scatter factor/hepatocyte growth factor (HGF), granulocyte/macrophage colony stimulating factor (GMCSF), a glial growth factor (GGF), and a fibroblast growth factor (FGF). The most preferred growth factors is EGF.

The liposomes can be used to administer a toxin, such as a botulinum toxin. Botulinum neurotoxin (BoNT) refers to botulinum serotypes A, B, C, D, E, F, G and all modified, substituted or fragment versions of these toxins that have a blocking effect on snare proteins. These include any substitution or modification of at least 1 amino acid of a naturally produced toxin or synthetically produced toxins. These modifications can be made with recombinant techniques. Also included are toxins with removal or substitution of the binding domain and/or translocation domain. Some of these variations of BoNT types A to G are discussed in U.S. Pat. No. 7,491,799 and by Bland et al. (Protein Expr. Purif, 71(1):62-73 (2010)). BoNT has been used effectively to treat different conditions with muscular hypercontraction.

The liposomes may also include anti-infectives, such as drugs to treat infections caused by bacteria, fungus, or viruses, analgesics, anti-inflammatories, anti-ulcer medications, antispasmodics, or other drugs used to treat gastric conditions.

Exemplary diagnostic agents which can also be included in the liposomes are, without limitation, paramagnetic molecules, fluorescent compounds, magnetic molecules, radionuclides, and x-ray imaging agents, and MRI contrast agents. Other agents can also be anti-inflammatories, angiogenesis inhibitors, and chemotherapeutic agents.

In certain embodiments, only one active agent is incorporated into the metastable liposome particles. Preferably, the active agent is hydrophobic, as demonstrated in the Examples. In other embodiments, two or more active agents are incorporated within the metastable liposomal particles.

C. Carriers and Excipients

The liposomes may be formulated with a pharmaceutically acceptable carrier and/or excipient for administration to tissue or a tissue lumen. Suitable carriers include, but are not limited to, sterile liquids, such as water, saline and phosphate buffered saline, and aqueous or water soluble gels such as polyvinyl pyrrolidone, alginate, and hyaluronic acid.

The formulations can optionally contain suitable amounts of wetting or emulsifying agents, or pH buffering agents.

Generally, the liposomes are supplied either separately or mixed together in unit dosage form, for example, as a dry lyophilized powder or water-free concentrate (lyophilisate) in a hermetically sealed container, such as an ampoule or sachet indicating the quantity of active agent. Where the formulation is to be administered by instillation, it can be dispensed with an instillation bottle containing sterile pharmaceutical grade water or saline.

### III. Methods of Manufacturing

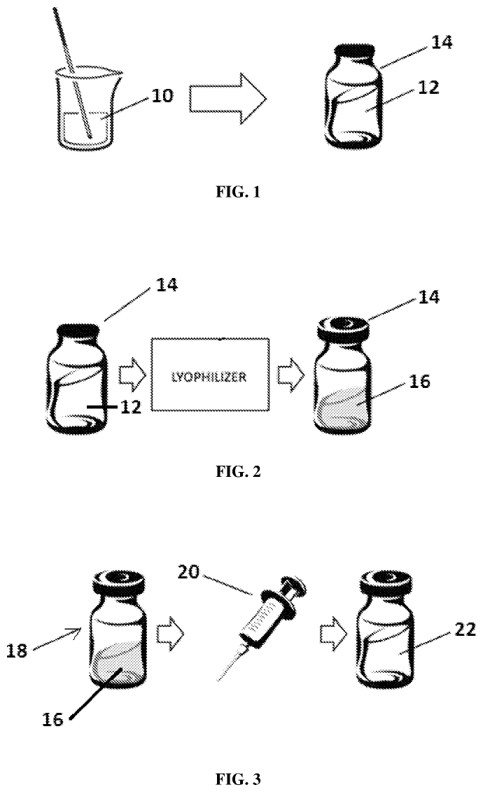

In certain instances, dehydrated/lyophilized liposomes are prepared as a pre-liposomal lyophilisate, such as shown in FIGS. 1 and 2. The pre-liposomal lyophilisate can be prepared using a method including the steps of:
(1) forming a solution of a sphingophospholipid and/or metabolites thereof and a surfactant in a water and tert-butyl alcohol (TBA) co-solvent system;
(2) lyophilizing the solution of step (1) to form a pre-liposomal lyophilisate.

In some instances, following step (1) and before step (2), the solution is aliquoted into suitable containers, such as single dose vials. The pre-liposomal lyophilisate formed in step (2) may be in the form of a pre-liposomal lyophilisate cake and may be formed directly in a vacuum sealed container, such as a single dose vial.

In some instances, the concentration of the sphingophospholipid, which is preferably sphingomyelin and/or metabolites thereof, used to form the pre-liposomal lyophilisate can have a concentration of the one or more sphingophospholipids in a range from between about 5 to 15 mg/mL or about 8 to 12 mg/mL in the co-solvent system. In some instances, the concentration of the sphingophospholipid, which may be sphingomyelin, is about 5, 6, 7, 8, 9, 10, 11, 12, 13, 14, or 15 mg/mL in the co-solvent system. Sphingomyelin metabolites can include, without limitation, ceramide, sphingosine, or sphingosine 1-phosphate.

In some cases, the water and tert-butyl alcohol (TBA) co-solvent system has a ratio of water-to-TBA ranging from about 40% to about 70%, or 50% to about 60% by volume/volume. In some instances, the ratio of water-to-TBA is preferably about 60% v/v.

In some instances, the surfactant is a sterol, such as cholesterol. In addition to cholesterol, other surfactants can include propylene glycol (PG). Combinations of surfactants may be used. In certain cases, the ratio of the surfactant(s) to sphingophospholipid ranges from about 1.5 to 10%, about 1.5 to 7.5%, about 1.5 to 5%, about 1.5 to 3%, or about 1.75 to 2.5% on a mass basis. In certain instances, the ratio of the surfactant(s) to sphingophospholipid is about 1.5%, 1.6%, 1.7%, 1.8%, 1.9%, 2%, 2.1%, 2.2%, 2.3%, 2.4%, 2.5%, 2.6%, 2.7%, 2.8%, 2.9%, or 3% on a mass basis.

Methods and conditions for lyophilization are known in the art.

As shown in FIG. 3, the pre-liposomal lyophilisate discussed above can be rehydrated to form a liposome-containing formulation by a method including the steps of:
(1′) combining an aqueous solution comprising one or more therapeutic, prophylactic, and diagnostic agents with a pre-liposomal lyophilisate (as described above) to form a liposomal formulation.

It is believed that the addition of the agent-containing aqueous solution to the pre-liposomal lyophilisate induces formation of liposomes having the therapeutic, prophylactic, and diagnostic agents associated therewith. The liposomal formulation formed can be a homogeneous suspension.

Exemplary therapeutic, prophylactic, and diagnostic agents are discussed above. In certain embodiments, one or more therapeutic, prophylactic, and diagnostic hydrophilic agents are used in the aqueous solution. In some embodiments, the liposomes include a hydrophilic agent which is immunoglobulin, such as an anti-PD-1, which can be effective for treating bladder diseases, such as cancer.

It is possible to maximize both the sphingophospholipid, such as sphingomyelin, concentration and the average, on a by-volume basis, liposome particle-size found in the homogeneous suspension formed in step (1′) when the pre-liposomal lyophilisate is reconstituted/rehydrated. This provides a homogeneous suspension that is stable to settling, meaning it does not settle (i.e. vertically separate) over time (resulting from exposure to a gravitational force of Ig). Settling can be determined by visual inspection of the homogeneous suspension and stability is considered no discernible settling for a period of at least four hours. In some instances, the homogeneous suspension is stable to settling for at least 4 hours, 10 hours, 15 hours, 20 hours, 1 day, 5 days, 10 days, 20 days, 30 days, or longer once formed. Further, the ratio of surfactant to sphingophospholipid can be minimized given the homogeneity constraint described herein.

In some instances, the volume of the aqueous solution used is about 25 to 100 mL, 25 to 75 mL, or 25 to 50 mL, or any sub-ranges within each. In some instances, the concentration of the one or more therapeutic, prophylactic, or diagnostic agents present in the aqueous solution can be in a concentration of about 0.05 mg/ml to 10 mg/ml, 0.05 mg/ml to 5 mg/ml, 0.05 mg/ml to 2.5 mg/ml, 0.05 mg/ml to 1 mg/ml, or 0.05 mg/ml to 0.75 mg/ml. A preferred agent in the aqueous solution is an immunoglobulin such as an anti-PD-1.

In some instances, the aqueous solution is made of sterile pharmaceutical grade water or saline and the one or more therapeutic, prophylactic, and diagnostic agents. In some cases, the aqueous solution may also contain other suitable solvents. For example, the aqueous solution can contain a mixture of water and an alcohol. Suitable alcohols include, but are not limited to, methanol, ethanol, propanol, (such as isopropanol), butanol (such as n-butanol, isobutene, sec-butanol, tert-butanol, pentanol (such as amyl alcohol, isobutyl carbinol), hexanol (such as 1-hexanol, 2-hexanol, 3-hexanol), heptanol (such as 1-heptanol, 2-heptanol, 3-heptanol and 4-heptanol) or octanol (such as 1-octanol) or a combination thereof.

The liposomal formulation formed in step (1′) is pharmaceutical formulation which is suitable for administration to a patient in need thereof, such as by instillation, and other methods of treatment described below.

### IV. Methods of Treatment with Liposome Formulations

Incorporation of agents, such as hydrophilic agents, into the lipid components of liposomes increases availability during instillation of liposome-containing formulation. Localized delivery has the advantage of reducing severe adverse effects associated with systemic delivery. One advantage to using the liposomes described, is the ability to deliver hydrophilic agents, such as anti-PD-1 (programmed cell death protein 1) immune checkpoint inhibitor “anti-PD-1,” to the bladder lumen, for example. Local delivery of hydrophilic agents, such as anti-PD-1 (programmed cell death protein 1) immune checkpoint inhibitor to the bladder lumen, may be both safer and more effective than current systemic anti-PD-1 approaches for the treatment of bladder cancer.

The liposome formulations described can be administered directly to the tissue or instilled into a tissue lumen. Representative tissue lumens include those of the respiratory, gastrointestinal, and urogenital tracts. These include cavities such as the nasal, pulmonary, esophageal, rectal, bladder, vaginal, urethral, and uterine cavities. In one embodiment the liposomes are suspended in a liquid formulation and spray or painted onto a tissue or instilled into a lumen for an effective amount of time, typically 30 to 60 minutes. In some instances, the liposome formulations are instilled via a Foley catheter. In some instances, the liposome formulations containing a suitable agent can be administered to a desired location in the bladder, other body cavity, or skin by spraying, rolling, painting or sponging a liquid, viscous liquid or gel-like material using a cystoscopy, endoscope, or other suitable scope device. The use of a scope device allows identification of the area of administration before administering the formulation. The scope device can include an applicator for the formulation including, but not limited to, a spraying device, gauze, roller or sponge containing the formulation. The applicator can be protected using a suitable cover until the formulation is to be administered so the formulation is not accidentally applied to an undesired area. The applicator can be attached at the end of the scope device to allow high precision administration. Liquid spray tools for scope devices are known in the art, for example such tool is described in U.S. Pat. Nos. 7,588,172 and 6,354,519 to Yamamoto and Kidooka.

The liposome encapsulated hydrophilic agent(s) are preferably administered by instillation into the bladder. In some instances, intravesical Bacillus Calmette-Guerin (BCG) is instilled with the liposomes. In some instances, the pre-liposomal lyophilate disclosed is to be rehydrated in pure immunoglobulin-G (Ig-G), without BCG present. This can result in a clinical situation with two separate instillations, which can be given the same day or on different days, as needed, of Ig-G (checkpoint inhibitor) and BCG. However, in some instances, it is possible to combine both in a single instillate administered to a patient in need thereof. Methods of instillation are known. (Lawrencia, et al., Gene Ther., 8:760-8 (2001); Nogawa, et al., J. Clin. Invest., 115:978-85 (2005); Ng, et al., Methods Enzymol., 391:304-13 (2005); Tyagi, et al., J. Urol., 171:483-9 (2004). (Trevisani, et al., J. Pharmacol. Exp. Ther., 309:1167-73 (2004); Trevisani, et al., Nat. Neurosci., 5:546-51 (2002)).

In the preferred embodiments, the liposomal formulation is administered by direct injection into the bladder wall. Any suitable number of injection sites, such as 5-20 sites, may be given to the subject in need thereof.

The selected dosage depends upon the desired therapeutic effect, on the route of administration, and on the duration of the treatment desired. Generally, dosage levels of about 1 to 10 mg/kg of body weight daily are administered to mammals, such as humans. In some instances, the preferred route of administration is intra-bladder administration.

Different size dosage units of the liposome-containing formulations may be used. A dosage unit containing a dry powder of a pre-liposomal lyophilisate which can be reconstituted in a container with a pharmaceutically acceptable carrier and a desired drug agent(s), as described above. Preferably, the pharmaceutically acceptable carrier is an aqueous carrier. Suitable amounts of hydrophilic agents in the formulations can be between about 0.1-1 mg, 1-3 mg, 3-10 mg, 10-20 mg or 20-50 mg. Suitable concentrations of the hydrophilic agents in the aqueous core of the liposomes include, but are not limited to, between about 0.05 mg/ml to 10 mg/ml, 0.05 mg/ml to 5 mg/ml, 0.05 mg/ml to 2.5 mg/ml, 0.05 mg/ml to 1 mg/ml, or 0.05 mg/ml to 0.75 mg/ml.

In a preferred embodiment, a bladder disorder is treated by instillation of agent in the liposomes, in an amount providing relief from the bladder disorder or one or more symptoms of the bladder disorder for a period of greater than a week, two, three, or four weeks, one, two or three two months, preferably greater than 6 months, following administration of the liposome formulation where the relief does not decline for a prolonged period of time relative to current therapies.

The liposome formulation can be administered one or more times to provide effective relief from one or more bladder disorder or symptoms associated with bladder disorders. Representative bladder disorders include interstitial cystitis (IC) and painful bladder (PBS), defined as a pain, pressure, or discomfort in the suprapubic or bladder area which can cause urinary frequency or the urge to urinate that has been present for at least six weeks. Other representative bladder disorders that can be treated with the formulations include hemorrhagic cystitis and cancer. Symptoms that can be alleviated by treatment with the liposome formulations describe include, but are not limited to, hematuria, urinary urgency, supra pubic pain, inflammation, and urinary retention.

The liposomal formulations can also be used to treat disorders of other parts of the body including, but not limited to, oropharyngeal parts, the mouth, the vagina, gastro-intestinal tract (upper and lower), such as the colon and/or rectum, airway, esophagus, nasal cavity, ear canal, and skin.

The present invention will be further understood by reference to the following non-limiting examples.

### Examples

### Example 1: Preparation of Liposomes

72 mg of pure sphingomyelin (SM) and 8 mg of cholesterol (CH) were dissolved in 40 mL of a water to tertiary-butyl alcohol (TBA) mixture. The mixture had a TBA/H₂O ratio of approximately 60%. The ratio of cholesterol to SM was approximately 2.3% on a mass basis.

FIG. 1 is a schematic of a solution 10 of sphingomyelin (SM) and cholesterol (CH) in a miscible mixture of tertiary butyl alcohol (TBA) and water (H₂O) on the left and on the right an aliquot 12 of solution 10 in a single-dose container 14.

Next, the resulting solution was lyophilized according to the following parameters The solution was lyophilized by first freezing at −40° C. for 30 min, then primary drying at 10° C. for 20 h under a vacuum of 200 micron, followed by secondary drying at 20° C. for 4.5 h. The lyophilisate produced was maintained in a vacuum-sealed vial.

FIG. 2 is a schematic of the transition of an aliquoted solution 12 via a lyophilization step to afford a pre-liposomal lyophilisate 16 in a vacuum-sealed container 14.

FIG. 3 is a schematic of the rehydration of a vacuum-sealed lyophilisate 16 by addition of an aqueously dissolved immunoglobulin 20, such as an anti-PD-1, resulting in formation of a liposomal immunoglobulin suspension 22 having an optimized immunoglobulin association with respect to the resulting therapeutic immunoglobulin potency.

Finally, an aqueous immunoglobulin-containing liposomal solution with an approximate 0.75 mg/mL concentration was drawn into the sterile syringe and the filled syringe was used in preparation of a liposomal immunoglobulin intravesical formulation, which was then drawn into a sterile 50 mL syringe used for urinary catheter instillation. The volume of suspension 22 of FIG. 3 was approximately 50 mL and 40 mL, respectively. These volumes optimize the instillation procedure for human patients, including patients who may have limited urinary bladder capacity.

The aforementioned parameters can be used to optimize potency of an intravesical instillate by maximizing both: (a) the SM concentration and (b) the average, on a by-volume basis, liposome particle-size in suspension. This provides a homogeneous suspension that is stable to settling, meaning it does not settle (i.e. vertically separate) over time (resulting from exposure to 1 g). Further, the ratio of CH to SM is minimized given the homogeneity constraint described herein.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A pre-liposomal lyophilisate in the form of a dry lyophilized powder for reconstitution to form a liposomal dosage formulation, wherein the liposomes, formed by reconstitution with an aqueous solution, comprise:
a shell comprising a sphingophospholipid and/or metabolites thereof;
a surfactant;
an aqueous core; and
one or more hydrophilic therapeutic, prophylactic or diagnostic agents encapsulated within the aqueous core of the liposomes.

2. The pre-liposomal lyophilisate of claim 1, wherein the dry lyophilized powder is a cake optionally present in a vacuum sealed container, such as a single dose vial.

3. The pre-liposomal lyophilisate of claim 1, wherein the sphingophospholipid is sphingomyelin.

4. The pre-liposomal lyophilisate of claim 1, wherein the surfactant is cholesterol.

5. The pre-liposomal lyophilisate of claim 1, wherein the surfactant to sphingophospholipid and/or metabolites thereof are at a ratio in a range from about 1.5 to 10%, about 1.5 to 7.5%, about 1.5 to 5%, about 1.5 to 3%, or about 1.75 to 2.5% on a mass basis.

6. The pre-liposomal lyophilisate of claim 1, wherein the aqueous solution used to reconstitute the pre-liposomal lyophilisate preferably is a sterile pharmaceutical grade water or saline solution or a mixture thereof with alcohol and comprises the one or more hydrophilic therapeutic, prophylactic or diagnostic agents.

7. The pre-liposomal lyophilisate of claim 6, wherein the one or more hydrophilic therapeutic, prophylactic or diagnostic agents are present in the aqueous solution at a concentration of about 0.05 mg/ml to 10 mg/ml, 0.05 mg/ml to 5 mg/ml, 0.05 mg/ml to 2.5 mg/ml, 0.05 mg/ml to 1 mg/ml, or 0.05 mg/ml to 0.75 mg/ml.

8. The pre-liposomal lyophilisate of claim 1, wherein the one or more hydrophilic therapeutic, prophylactic or diagnostic agents encapsulated within the aqueous core of the liposomes comprise immunoglobulin.

9-10. (canceled)

11. The pre-liposomal lyophilisate of claim 6, wherein the alcohol is selected from the group consisting of methanol, ethanol, propanol, (such as isopropanol), butanol (such as n-butanol, isobutene, sec-butanol, tert-butanol, pentanol (such as amyl alcohol, isobutyl carbinol), hexanol (such as 1-hexanol, 2-hexanol, 3-hexanol), heptanol (such as 1-heptanol, 2-heptanol, 3-heptanol and 4-heptanol), octanol (such as 1-octanol), and combinations thereof.

12. A pharmaceutical dosage formulation comprising the lyophilisate of claim 1 liposomes, wherein the liposomes comprise:
a shell comprising a sphingophospholipid and/or metabolites thereof;
a surfactant;
an aqueous core; and
one or more hydrophilic therapeutic, prophylactic or diagnostic agents encapsulated within the aqueous core of the liposomes.

13-20. (canceled)

21. The pharmaceutical dosage formulation of claim 12, wherein the pharmaceutical dosage formulation is a homogeneous suspension that is stable to settling for at least 1 day, 5 days, 10 days, 20 days, or 30 days.

22. The pharmaceutical dosage formulation of claim 12, wherein the liposomes are suitable for direct administration to a tissue or tissue lumen, preferably bladder for the treatment of a bladder disease or disorder.

23. (canceled)

24. The pharmaceutical dosage formulation of claim 12, wherein the liposomes have an average diameter in a range of between about 0.1 to 50 microns.

25. A method of preparing a pre-liposomal lyophilisate in the form of a dry lyophilized powder for reconstitution to form a liposomal dosage formulation, the method comprising the steps of:
(1) forming a solution of a sphingophospholipid and/or metabolites thereof and a surfactant in a water and tert-butyl alcohol (TBA) co-solvent system;
(2) lyophilizing the solution of step (1) to form the pre-liposomal lyophilisate,
preferably wherein following step (1) and before step (2), the solution is aliquoted into a suitable container, such as a single dose vial.

26. (canceled)

27. The method of claim 25, wherein the pre-liposomal lyophilisate formed in step (2) forms a pre-liposomal lyophilisate cake optionally formed directly in a vacuum sealed container, such as a single dose vial.

28. The method of claim 25, wherein the concentration of the sphingophospholipid and/or metabolites thereof in the solution is in a range from between about 5 to 15 mg/mL or about 8 to 12 mg/mL in the co-solvent system.

29. The method of claim 25, wherein the water and tert-butyl alcohol (TBA) co-solvent system has a ratio of water-to-TBA ranging from about 40% to about 70% or 50% to about 60% by volume/volume.

30. The method of claim 29, wherein the ratio of water-to-TBA is about 60% volume/volume.

31-33. (canceled)

34. A method of preparing a liposomal dosage formulation by reconstitution of the pre-liposomal lyophilisate of claim 12, the method comprising the steps of:
(1′) combining an aqueous solution comprising one or more hydrophilic therapeutic, prophylactic, and diagnostic agents with the pre-liposomal lyophilisate to form a liposomal dosage formulation.

35-46. (canceled)

47. A method for treating an individual in need thereof comprising administering to a tissue or tissue lumen, preferably bladder, the pharmaceutical dosage formulation of claim 25.

48. The method of claim 47, wherein the pharmaceutical dosage formulation is administered via instillation.

49. The method of claim 47, wherein the pharmaceutical dosage formulation is administered via a cystoscope comprising an applicator selected from the group consisting of a spray device, gauze, roller, and sponge.

50. The method of claim 47 comprising administering the pharmaceutical dosage formulation to a lumen selected from the group consisting of lumens of the respiratory tract, the gastrointestinal tract, the urino-genital tract, and the reproductive tract.

51. The method of claim 47, wherein the treatment is of a bladder disease or disorder, and the method comprises administering the pharmaceutical dosage formulation in an effective amount to an individual with a bladder disease or disorder.

52. The method of claim 51, wherein the bladder disease or disorder is selected from the group consisting of hemorrhagic cystitis, interstitial cystitis/painful bladder syndrome, and bladder cancer.

53. The method of claim 51, wherein the pharmaceutical dosage formulation is administered via instillation into the bladder or injection directly into the bladder.
